(19) United States
(12) Patent Application Publication
(10) Pub. No.: US 2019/0209037 A1
(43) Pub. Date: Jul. 11, 2019

(54) ECG ANALYSIS FOR DIAGNOSIS OF HEART FAILURE AND CARDIOVASCULAR DISEASE USING SIGNALS OBTAINED FROM AN IMPLANTABLE MONITOR

(71) Applicant: BIOTRONIK SE & CO. KG, Berlin (DE)

(72) Inventor: JOSEPH SIMON RAVEN, TIGARD, OR (US)

(21) Appl. No.: 16/239,127

(22) Filed: Jan. 3, 2019

Related U.S. Application Data
(60) Provisional application No. 62/615,021, filed on Jan. 9, 2018.

(51) Int. Cl. A61B 5/0456; A61B 5/042; A61B 5/0472; A61B 5/0432

(52) U.S. Cl. CPC A61B 5/0456; A61B 5/0432; A61B 5/0472; A61B 5/042

(57) ABSTRACT

An implantable device for monitoring of a patient's electrocardiogram, has a detection element for determining electrocardiogram signals of a heart of the patient. The signals are indicative of the electrocardiogram of the patient. The implantable device includes a processor that is configured to determine from the signals at least one parameter of the electrocardiogram. The at least one parameter is an R:S ratio defined as the ratio between the absolute value of the R complex and the absolute value of the S complex.

### CROSS-REFERENCE TO RELATED APPLICATION

This application claims the benefit, under 35 U.S.C. § 119(e), of provisional patent application No. US 62/615,021 filed Jan. 9, 2018; the prior application is herewith incorporated by reference.

### BACKGROUND OF THE INVENTION

### Field of the Invention

The disclosure relates to an implantable device for monitoring the heart of a patient.

Worldwide the leading causes of death among non-communicable diseases are cardiovascular diseases, many of which contribute to, or are the result of, heart failure (HF). The World Health Organization (WHO) Global Health Observatory data repository indicates that nearly 1.8 million deaths will occur as the result of cardiovascular disease (CVD) and this number is projected to climb to 22.2 million by 2030.

United States patent application No. US 2004/0116819 A1 discloses an implantable monitoring device which measures and stores the patient's electrocardiogram (ECG) and respiration data via electrodes on the surface of the device.

ECG analysis often relies on monitoring by physicians or health care providers or on self-monitoring of the patient to indicate a change in the progression of the patient's disease and the efficacy of their treatment.

In particular, the frequency of ECG data available to physicians on the state of their patient regarding the progression of CVD or HF may be limited to the number of visits and surrounding environmental conditions of the visits made by a patient.

### SUMMARY OF THE INVENTION

Therefore, it is an objective to provide an implantable cardiac monitoring device that helps in generating prognostic indicators of worsening or imminent CVD and HF, as well as providing more accurate diagnosis concerning the progression of CVD/HF of the individual patient.

With the above and other objects in view there is provided, in accordance with the invention, an implantable device for monitoring a patient's electrocardiogram, the device comprising:

a detection element for acquiring electrocardiogram signals of a heart of the patient, the signals being indicative of the electrocardiogram of the patient;

a processor connected to said detection element and configured to determine from the signals at least one parameter of the electrocardiogram, the at least one parameter being an R:S ratio defined as a ratio between an absolute value of an R complex and an absolute value of an S complex.

In other words, the novel implantable device for monitoring a patient's ECG includes a detection element for determining electrocardiogram signals of a heart of the patient when the device is surgically implanted in the (human or animal) patient, wherein signals are indicative of the electrocardiogram of the patient, wherein the implantable device (also denoted as cardiac monitoring device) comprises a processor that is configured to determine from the signals at least one parameter of the electrocardiogram or a quantity derived from the at least one parameter/ECG, particularly for: (1) providing prognostic indicators of disease development or early indicators of risk for developing CVD, (2) for diagnosing various cardiovascular diseases or heart failure HF or for determining a progression relating to CVD/HF. The detection element may be a lead.

Thus, utilizing an implantable cardiac monitor, the invention allows assessing various ECG parameters over time, particularly using various algorithms and metrics to generate data which can be made available to the clinician to facilitate the management of patients at risk of, or with, HF or CVD.

The device disclosed herein allows for more information regarding patient disease progression on the basis of more frequent sampling and analysis not normally available to the physician. The types of analyses described herein can efficiently be made by an implantable medical device with the capability of monitoring and recording subcutaneous ECGs thereby providing valuable diagnostic information which has prognostic and diagnostic relevance and provides improved resolution on treatment efficacy.

According to an embodiment, the at least one parameter is a QRS amplitude of the electrocardiogram (ECG).

Particularly, the analysis of the QRS amplitude (i.e. the time interval or duration of the QRS complex) can be utilized as a diagnostic tool for, and an indicator of, Left Ventricular End Diastolic Diameter (LVEDD), Left Ventricular End Systolic Diameter (LVESD), Amylosis, Lower Limb Edema (LLE), and can also be used as an indicator of fibrosis.

Therefore, according to an embodiment, the processor is configured to record a plurality of successive QRS amplitudes QRS₍ᵢ₋ₜ₎ during a first sampling period and a plurality of successive QRS amplitudes QRS₍ᵢ₎ during a later second sampling period, and to determine a net change of the mean QRS amplitudes Δ̅Q̅R̅S̅ between the two sampling periods using the relation Δ̅Q̅R̅S̅=Q̅R̅S̅₍ᵢ₎−Q̅R̅S̅ᵢ₋ₜ), wherein Q̅R̅S̅₍ᵢ₎ is the average QRS amplitude of the second sampling period, and wherein Q̅R̅S̅₍ᵢ₋ₜ₎ is the average QRS amplitude of the earlier first sampling period.

Particularly, according to an embodiment, in instances where Δ̅Q̅R̅S̅ is negative, the negative value is indicative that factors influencing QRS amplitude like LVEDD (Left Ventricular End Diastolic Diameter), LVESD (Left Ventricular End Systolic Diameter), LVH (left ventricular hypertrophy), LLE (Lower Limb Edema), or Amylosis may develop or worsen and therefore the patient has a poor or worsening prognostic outlook. This uses the assumption that the QRS amplitude will be negatively correlated with the severity of the diseased state.

Furthermore, in an embodiment, the processor, or processing unit, is configured to attribute a negative net change of the QRS amplitude (Δ̅Q̅R̅S̅) to at least one of: LVEDD (Left Ventricular End Diastolic Diameter), LVESD (Left Ventricular End Systolic Diameter), LVH (left ventricular hypertrophy), LLE (Lower Limb Edema), or Amylosis.

Further, according to an embodiment, the device is configured to communicate information/data comprising the net change of the mean QRS amplitude (Δ̅Q̅R̅S̅), and particularly the attribution, via a telemetry unit of the device to a remote external device (e.g. a device that is used by the patient, a physician or a health care provider to obtain the information data, e.g. for purposes of diagnosis etc.). Such an external device can be a smart phone, a computer, a remote server etc. Here (as well as in the other embodiments), the device may merely provide the information or data and leave attributing this data to a certain CVD/HF to the external device, or medical practitioners operating the external device.

Further, according to an embodiment, the processing unit is configured to determine and record a plurality of successive net changes of the mean QRS amplitude Δ̅Q̅R̅S̅ as a function of time, particularly from the time at which the cardiac monitoring device has been implanted and started for the first time until the time at which the net change of the mean QRS amplitude Δ̅Q̅R̅S̅ has been determined most recently.

Particularly, according to an embodiment, the implantable cardiac monitoring device is configured to communicate information/data comprising the plurality of net changes of the QRS amplitude to the external device, particularly for purposes of prognosis/diagnosis. Particularly, the external device may be used to graphically display the net changes for further analysis of the latter. This can be used to provide diagnostic information about the efficacy of treatment and patient prognosis/diagnosis.

Furthermore, R-R variability has longed been recognized as an indicator of the health or tone of the autonomic nervous system and may serve as an indicator in the progression of CVD/HF. Therefore, according to a further embodiment, the at least one parameter is an R-R duration of the electrocardiogram (ECG).

Particularly, according to an embodiment, the processing unit is configured to determine and record a plurality of successive R-R durations Rdᵢ.

Further, according to an embodiment, the processing unit is configured to determine a measure R̅d̅, particularly a mean, of the plurality of R-R durations, wherein the processing unit is further configured to determine a variance S_{(Rd)}² of the measure using the relation

$${S}_{( Rd )}^{2} = \frac{1}{n - 1} {\sum }_{i = 1}^{n} {( {Rd}_{i} - \overline{Rd} )}^{2} ,$$

wherein n is the number of recorded R-R durations, Rdᵢ is the i-th recorded R-R duration, and R̅d̅ is the mean of the plurality of Rdᵢ samples.

Furthermore, in an embodiment, the processing unit is configured to determine and record a plurality of variances S_{(Rd)}² for measures of R-R variance across time.

Further, according to an embodiment, the processing unit is configured to determine and record at least one or a plurality of F values according to

$$F = \frac{{S}_{Rd ( α )}^{2}}{{S}_{Rd ( β )}^{2}} ,$$

wherein S_{Rd(α)}² is a variance of the plurality of variances that has been determined more recently than the variance S_{Rd(β)}², which is a variance of the plurality of variances, too. Particularly S_{Rd(β)}² is the initial variance determined and recorded first.

In an embodiment, the device is configured to allow for selecting/programming the time between the earlier and the more recent variance used to determine the at least one F value (or the plurality of F values).

Particularly, F values greater than one indicate an increase in R-R variability which may indicate an improvement in patient prognosis, whereas values less than one indicate a negative prognosis. Values of 1 are diagnostic of no observed change.

Thus, in an embodiment, the processing unit is configured to attribute F values greater than one to an improvement in patient prognosis concerning CVD/HF, and to attribute F values less than one to a negative prognosis concerning CVD/HF. Under these conditions a noise floor or assessment of statistical significance may be selectively programmed, non-selectively programmed, or omitted to determine the magnitude of change, above or below zero, to be considered as a clinically relevant measure.

Further, in an embodiment, the device is configured to communicate information data comprising the at least one F value or the plurality of F values, and particularly the attribution, via the telemetry unit of the device to the external device, e.g. for purposes of diagnosis (see also above). Particularly, the F value(s) may be graphically displayed using the external device, e.g. for the purpose of diagnoses etc.

Further, according to an embodiment, the processing unit is configured to determine and record a difference ΔF between two of the recorded F values Fx and Fy according to ΔF=Fy−Fx, wherein Fx has been determined and recorded before Fy, and wherein particularly the processing unit is configured to determine and record a plurality of such differences of recorded F values.

In an embodiment, the device is configured to allow for selecting and/or programming the time between the F value Fx and the respective more recent F value Fy, so that particularly a history of differences ΔF can be generated.

Specifically, the difference ΔF may provide an index on the progression of autonomic dysfunction over time by comparing two programmable/selectable results from the stored F values, e.g. Fx and Fy. Negative values for ΔF can be used as an index of poor prognosis and are diagnostic of a progression of CDV/HF.

Thus, in an embodiment, the processing unit is configured to attribute negative values for ΔF to a progression of CDV/HF. Under these conditions a noise floor or assessment of statistical significance may be selectively programmed, non-selectively programmed, or omitted to determine the magnitude of change, above or below zero, to be considered as a clinically relevant measure.

Further, in an embodiment, the device is configured to communicate information data comprising the at least one difference ΔF or the plurality of differences ΔF, and particularly the attribution, via the telemetry unit of the device to the external device, e.g. for purposes of prognosis/diagnosis (see also above). Particularly, the difference(s) ΔF may be graphically displayed using the external device, e.g. for the purposes of prognoses/diagnoses etc.

Furthermore, episodes and duration of tachycardia are known to increase as the heart attempts to make up for its mechanical failings; specifically compensatory rate changes to maintain a constant output. Tachycardia may also be tracked utilizing the variable Rd as well. However, instead of calculating a variance to the population of samples, a mean will be generated, as shown below.

Therefore, according to an embodiment, the processing unit is configured to determine and record a mean (R̅d̅₀̅), of the plurality of R-R durations according to

$${\overline{Rd}}_{o} = \frac{1}{n} ( {\sum }_{i = 1}^{n} {Rd}_{i} ) ,$$

wherein n is the number of recorded R-R durations, and Rdᵢ is the i-th recorded R-R duration.

Particularly, in an embodiment, the device is configured to allow for selecting/programming the number of recorded R-R durations (samples) or the sampling duration. However, the number (sampling duration) may also be fixed.

Further, according to an embodiment, the processing unit is configured to determine and record a plurality of means (R̅d̅₀̅) of R-R durations as a function of time.

Furthermore, according to an embodiment, the processing unit is configured to determine and record a difference ΔRd between two of the recorded means R̅d̅ₓ and R̅d̅_{y} according to ΔRd=R̅d̅_{y}−R̅d̅ₓ, wherein R̅d̅ₓ has been determined and recorded before R̅d̅_{y}, and wherein the processing unit is configured to determine and record a plurality of such differences ΔRd of recorded means.

In an embodiment, the device is configured to allow for selecting and/or programming the time between the means R̅d̅ₓ and R̅d̅_{y}, so that particularly a history of differences ΔRd can be generated and recorded.

Particularly, a value of the respective difference ΔRd being smaller than zero or a negative correlation between time and the differences ΔRd₀ may be diagnostic of a worsening CVD/HF as well as tachycardia alone. Thus, in an embodiment, the processing unit may be configured to attribute negative values for ΔRd as well as a negative relationship between time and R̅d̅₀ to a progression of Tachycardia, CVD, or HF. Under these conditions a noise floor or assessment of statistical significance may be selectively programmed, non-selectively programmed, or omitted to determine the magnitude of change, above or below zero, to be considered as a clinically relevant measure.

Further, in an embodiment, the device is configured to communicate information data comprising the at least one difference ΔRd or the plurality of differences ΔRd, and particularly the attribution, via the telemetry unit of the device to the external device, e.g. for purposes of prognosis/diagnosis (see also above). Particularly, the difference(s) ΔRd may be graphically displayed using the external device, e.g. for the purposes of prognoses/diagnoses etc.

Furthermore, Q-T interval elongation may be seen as an indicator of pump failure, electrical conduction changes, ischaemia, or ventricular aneurysm. Therefore, according to an embodiment, the at least one parameter is a Q-T duration.

Furthermore, according to an embodiment, the processing unit is configured to determine and record a plurality of successive or non-successive Q-T durations QTᵢ.

Particularly, this may be most easily implemented by measuring from the peak (absolute maximum for each complex) as well as other methods such as a start or end detection method. In the instance the Q wave is not present or has poor resolution the P-wave or R-wave may be utilized as a non-superior alternative. The number of samples or sampling duration may be a fixed or selectable/programmable feature.

Particularly, according to an embodiment, the processing unit is configured to determine a mean Q̅T̅₀ of the plurality of Q-T durations according to

$${\overline{QT}}_{o} = \frac{1}{n} ( {\sum }_{i = 1}^{n} {QT}_{i} ) ,$$

wherein n is the number of recorded Q-T durations, and QTᵢ is the i-th recorded Q-T duration.

Particularly, in an embodiment, the number of recorded Q-T duration (samples) or the sampling duration may be fixed or selectable/programmable.

Furthermore, according to an embodiment, the processing unit is configured to determine and record a plurality of means Q̅T̅₀ of Q-T durations as a function of time.

Further, according to an embodiment, the processing unit is configured to determine and record a difference ΔQT between two of the recorded means Q̅T̅ₓ and Q̅T̅_{y} according to ΔQT=Q̅T̅_{y}−Q̅T̅ₓ, wherein Q̅T̅ₓ has been determined and recorded before Q̅T̅_{y}, wherein particularly the processing unit is configured to determine and record a plurality of differences ΔQT between [respectively two] recorded means. Preferably, in an embodiment, the device is configured to allow for selecting/programming the time between the means Q̅T̅ₓ and Q̅T̅_{y}, so that desired selectable history of differences ΔQT can be generated.

Particularly, a value greater than zero for ΔQT, or a positive correlation between time and the means Q̅T̅₀, may be diagnostic of worsening CVD/HF. Under these conditions a noise floor or assessment of statistical significance may be selectively programmed, non-selectively programmed, or omitted to determine the magnitude of change, above or below zero, to be considered as a clinically relevant measure.

Thus, in an embodiment, the processing unit is configured to attribute positive values for ΔQT or a positive correlation between time and the means Q̅T̅₀ to a worsening of one of: pump failure, electrical conduction changes, ischaemia, or ventricular aneurysm.

Further, in an embodiment, the device is configured to communicate information data comprising the at least one difference ΔQT or the plurality of differences ΔQT, and particularly the attribution, via the telemetry unit of the device to the external device, e.g. for purposes of prognosis/diagnosis (see also above). Particularly, the difference(s) ΔQT may be graphically displayed using the external device, e.g. for the purposes of prognoses/diagnoses etc.

Finally, an R:S wave amplitude ratio may be utilized as an index of left ventricular hypertrophy (LVH) and right ventricular hypertrophy (RVH); which is in many cases a precursor and progressive indicator of CVD and HF. Thus, according to an embodiment, the at least one parameter is an R:S ratio (R:S) defined as the ratio between the absolute value of the R complex and the S complex, i.e.,

$$( R \text{:} S ) = \frac{\vert {R}_{peak} \vert}{\vert {S}_{peak} \vert} .$$

Particularly, according to an embodiment, the processing unit is configured to determine and record a plurality of successive R:S ratios (R:S).

Further, according to an embodiment, the processing unit is configured to determine a mean (̅R̅:̅S̅)̅₀ of the plurality of R:S ratios according to

$${( \overline{R \text{:} S} )}_{0} = \frac{1}{n} ( {\sum }_{i = 1}^{n} {( R \text{:} S )}_{i} ) ,$$

wherein n is the number of recorded R:S ratios, and (R:S)ᵢ is the i-th recorded R:S ratio.

Particularly, in an embodiment, the number of recorded R:S ratios (samples) or the sampling duration may be fixed or selectable/programmable.

Furthermore, according to an embodiment, the processing unit is configured to determine and record a plurality of means (̅R̅:̅S̅)̅₀ of R:S ratios as a function of time.

Particularly, in an embodiment, the processing unit is configured to determine a difference Δ(R:S) between two of the recorded means (̅R̅:̅S̅)̅ₓ and (̅R̅:̅S̅)̅_{y} according to Δ(R:S)=(̅R̅:̅S̅)̅_{y}−(̅R̅:̅S̅)̅ₓ, wherein (̅R̅:̅S̅)̅ₓ has been determined and recorded before (̅R̅:̅S̅)̅_{y}, wherein particularly the processing unit is configured to determine and record a plurality of such differences Δ(R:S) between recorded means.

Preferably, in an embodiment, the device is configured to allow for selecting/programming the time between the means (̅R̅:̅S̅)̅ₓ and (̅R̅:̅S̅)̅_{y}, so that particularly a history of differences Δ(R:S) can be generated.

In particular, devices implanted with the ECG axis oriented parallel to the midline or in a positive deviation toward the left of the midline but less than perpendicular to midline, will exhibit an increase in the R:S ratio over time in the event RVH is worsening, while the value will decrease from the time of implant if LVH is worsening. The same formulae and methodology associated with comparing values, as performed for Q-T elongation and R-R duration, may be utilized to provide this diagnostic data, i.e., a value greater than zero for the difference Δ(R:S) or a positive correlation between time and the means (̅R̅:̅S̅)̅₀ can be diagnostic of worsening of RVH, whereas a value smaller than zero for the difference Δ(R:S) or a negative correlation between time and the means (̅R̅:̅S̅)̅₀ can be diagnostic of worsening of LVH. Under these conditions a noise floor or assessment of statistical significance may be selectively programmed, non-selectively programmed, or omitted to determine the magnitude of change, above or below zero, to be considered as a clinically relevant measure.

Thus, in an embodiment, the processing unit is configured to attribute a value greater than zero for the difference Δ(R:S) or a positive correlation between time and the means (̅R̅:̅S̅)̅₀ to a worsening of RVH, and to attribute a value smaller than zero for the difference Δ(R:S) or a negative correlation between time and the means (̅R̅:̅S̅)̅₀ to a worsening of LVH.

Further, in an embodiment, the device is configured to communicate information data comprising the at least one difference Δ(R:S) or the plurality of differences Δ(R:S), and particularly the attribution, via the telemetry unit of the device to the external device, e.g. for purposes of prognosis/diagnosis (see also above). Particularly, the difference(s) Δ(R:S) may be graphically displayed using the external device, e.g. for the purposes of prognoses/diagnoses etc.

Particularly, the device can be configured to determine and record an arbitrary combination of the above-discussed parameters/quantities, particularly depending on the specific needs of the individual patient.

Other features which are considered as characteristic for the invention are set forth in the appended claims.

Although the invention is illustrated and described herein as embodied in a method and device for ECG analysis for diagnosis of heart failure and cardiovascular disease using signals obtained from an implantable monitor, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 shows a schematic representation of a device according to the invention and an electrocardiogram detected by the device;

FIG. 2 shows an algorithm for determining a difference (net change) between average QRS amplitudes carried out by a processing unit of the device;

FIG. 3 shows an algorithm for determining a variance of a measure for an R-R duration carried out by a processing unit of the device;

FIG. 4 shows an algorithm for conducting an F test and for determining a difference between F values;

FIG. 5 shows an algorithm for determining a difference (net change) between average R-R durations amplitudes carried out by a processing unit of the device;

FIG. 6 shows an algorithm for determining a difference (net change) between average Q-T durations carried out by a processing unit of the device; and

FIG. 7 shows an algorithm for determining a difference (net change) between average R:S ratios carried out by a processing unit of the device.

### DETAILED DESCRIPTION OF THE INVENTION

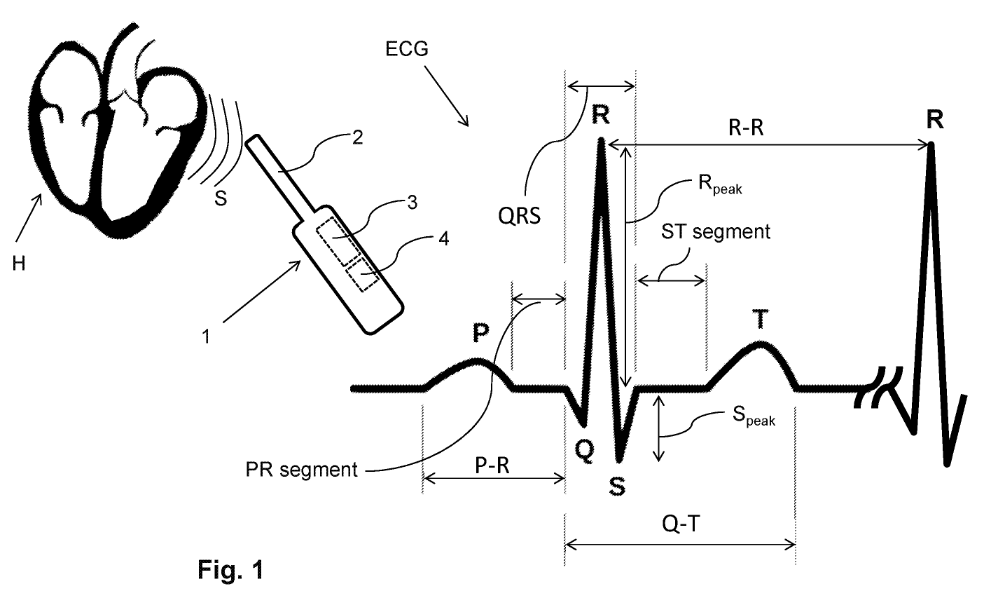

Referring now to the figures of the drawing in detail and first, particularly, to FIG. 1 thereof, there is shown an embodiment of an implantable device 1 for monitoring of a patient's electrocardiogram (ECG).

The device 1 comprises a detection element (e.g. a lead or a comparable suitable means) 2 for determining electrocardiogram signals S of a heart H of a human or animal patient when the device is implanted in the vicinity of the heart H. The device 1 thus forms a subcutaneous implantable device 1. The signals S are indicative of the electrocardiogram ECG of the patient, which ECG is indicated on the right hand side of FIG. 1. The ECG shows a P wave, QRS complex, a T wave, and a further succeeding QRS complex (the P wave is omitted here) in order to indicate an R-R interval. The U wave following the T wave is not shown. Also indicated is a P-R interval, a QRS amplitude (the sum of the Rₚₑₐₖ and Sₚₑₐₖ as indicated), QRS duration, and a Q-T inverval.

Furthermore, the cardiac monitoring device 1 includes a processor 3, or processing unit 3, that is configured to determine from the signals S at least one parameter of the electrocardiogram ECG or a quantity derived from such a parameter, the ECG, particularly for prognosis or diagnosing of CVD or HF or for determining a progress in HF/CVD.

Particularly, the processor 3 is configured to conduct algorithms for determining those parameters or quantities, particularly for monitoring CVD/HF as will be described in more detail below.

Further, the device 1 comprises a telemetry unit 4, via which the parameter and/or quantities generated by the processor 3 can be transmitted to a remote external device located outside the human or animal body (e.g. a smart phone, a computer, or a remote server), via which the parameters/quantities can be graphically displayed and/or further analyzed in order to prognose/diagnose and evaluate progression of CVD, HF, or other pathologies referenced previously, of the patient.

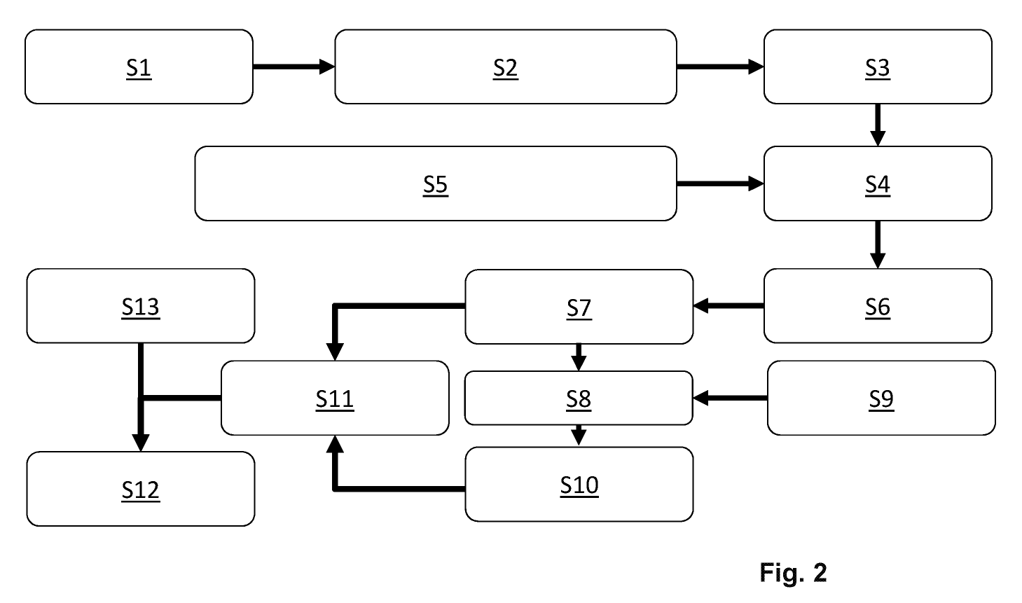

FIG. 2 illustrates an exemplary method for establishing the algorithm to determine a difference (net change) between average QRS amplitudes carried out by a processing unit 3. Here, the electrocardiogram signal is detected by the detection element 2 (step S1). The processing unit 3 detects the QRS complexes (QRS Amplitude Detection Algorithm, step S2) in the received ECG signals (S1) and forms corresponding output variables (QRS output variable, step S3) which are recorded (buffered outputs, step S4). The processing unit 3 further allows an operator to program/select (step S5) the sampling period (programmable sampling period (buffer size)), i.e., the number n of samples, or the period of time over which samples are averaged to determine (average calculation, step S6) an average QRS amplitude Q̅R̅S̅₍ᵢ₎ (step S7). The determined averages are recorded (buffered outputs, step S8), wherein in turn the number of recorded averages can be programmed/selected (programmable buffer size, step S9). From these recorded averages an earlier average is selected (prior output value Q̅R̅S̅₍ᵢ₋ₜ_{ₙ}₎, step S10) and a net change (difference) of the QRS amplitude ΔQRS is determined (final output, step S11) according to

Δ̅Q̅R̅S̅=Q̅R̅S̅₍ᵢ₎−Q̅R̅S̅₍ᵢ₋ₜ₎ Eq (1):

wherein (i) indicates the more recent sampling period over which QRS amplitudes have been collected and averaged, while (i-t) indicates the prior sampling period which may be fixed or programmed. The determined net changes ΔQRS in QRS amplitude over the time between the considered sampling periods are recorded/stored (stored history, step S12) to have a history of these values for later analysis, wherein the number of stored net changes ΔQRS can be programmed (programmable buffer size, step S13).

Equation (1) provides the means by which QRS amplitudes may be evaluated as an indicator of CVD/HF. Particularly, in instances where ΔQ̅R̅S̅ is negative, the negative value may suggest that factors influencing QRS amplitude like LVEDD, LVESD, LVH, LLE, or Amylosis may developing or worsening and therefore the patient has a poor or worsening prognostic outlook. This makes an educated assumption that QRS amplitude will be negatively correlated with the severity of the diseased state.

Additionally, by plotting or tracking the Q̅R̅S̅ or ΔQ̅R̅S̅ measurements over time (from implant to the most recent sample) one may be able to provide diagnostic information about the efficacy of treatment and patient prognosis.

Furthermore, Equations (2-1), (2-2), and (2-3) below provide the means by which R-R variability in the detected ECG (cf. FIG. 1) may be evaluated as an indicator of CVD/HF.

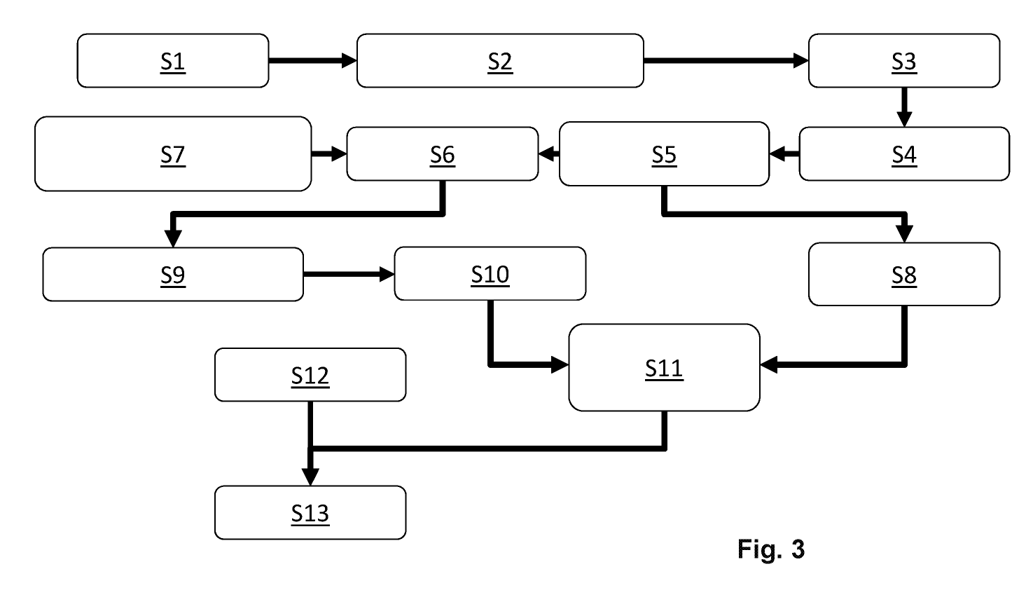

This is used in an embodiment according to FIG. 3, wherein the processing unit 3 is configured to conduct an algorithm for calculating a variance S_{(Rd)}² of a measure R̅d̅ for the R-R interval (duration) as indicated in FIG. 1.

For this, according to FIG. 3, the electrocardiogram signal S is detected by the detection element 2 (step S1), and the processing unit 3 detects the QRS complexes (QRS detection algorithm, step S2) in the received ECG signals S and uses time stamps for marking two successive R waves (R-wave marker time stamp, step S3), which are recorded (buffered outputs (2-sample), step S4) and used to determine the corresponding R-R interval (delta of outputs calculation (R-R interval), step S5), which is recorded (buffered delta outputs, step S6), wherein the number of recorded R-R intervals can be selected/programmed (programmable sampling period (buffer size), step S7).

The recorded R-R intervals (S6) are averaged (average calculation of deltas, step S9) to form a measure (e.g. average) R̅d̅ of the R-R intervals (output R̅d̅, step S10) which is used in conjunction with the individual R-R intervals (most recent delta Rdᵢ, step S8) that have been determined in step S5, respectively, in order to determine a variance of the measure R̅d̅ according to

$${S}_{( Rd )}^{2} = \frac{1}{n - 1} {\sum }_{i = 1}^{1} {( {Rd}_{i} - \overline{Rd} )}^{2} \qquad Eq ( 2 \text{-} 1 )$$

wherein n denotes the number of samples.

The resultant variance S_{(Rd)}² from Eq (2-1) is then recorded (stored history, step S13) for further analysis and tracking, wherein the number of stored variances S_{(Rd)}² can be selected/programmed (programmable buffer size, step S12).

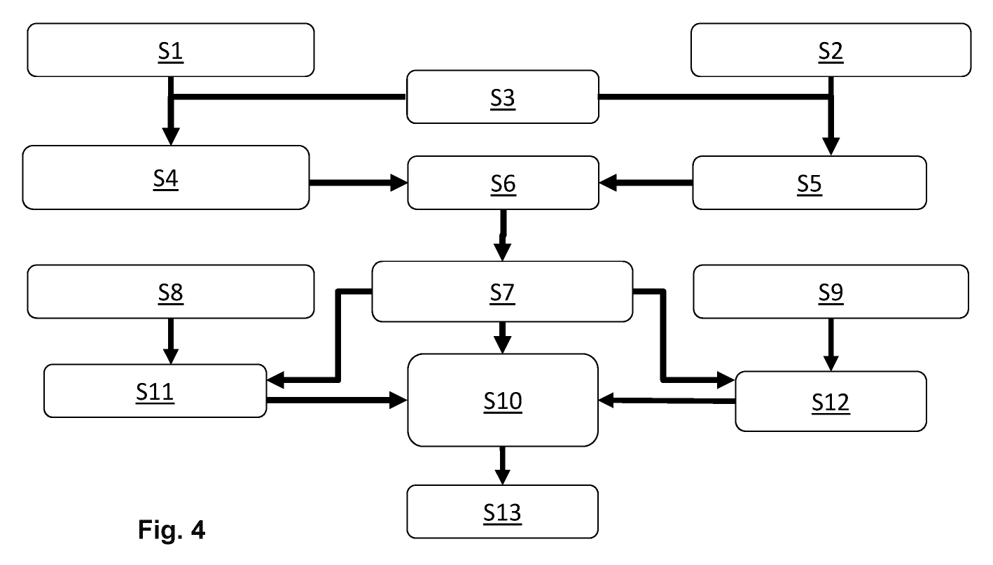

Further, according to an embodiment shown in FIG. 4, stored values (stored history S_{(Rd)}², S3) for the variance may then be compared to one another using an F-Test which may indicate the relative change in R-R variability across time. Here, more recent variance values S_{Rd(α)}² are used (S4) together with an earlier (or the earliest measured) value S_{Rd(β)}² (S5) in order to determine corresponding F values (F-test calculation, step S6) according to

$$F = \frac{{S}_{Rd ( α )}^{2}}{{S}_{Rd ( β )}^{2}} \qquad Eq ( 2 \text{-} 2 )$$

F values greater than one indicate an increase in R-R variability which may indicate an improvement in patient prognosis, whereas values less than one indicate a negative prognosis. Values of 1 are diagnostic of no observed change.

The time between each compared measure in Eq. (2-2) is particularly programmable/selectable (programmable sample period; S1, S2). Multiple comparisons may also be made and displayed for additional diagnostic and statistical data.

Additionally, as indicated in FIG. 4, the products of Eq. (2-2) may be stored (stored history, S7) and used for further analysis and tracking. Specifically, Equation (2-3) may provide an index on the progression of autonomic dysfunction over time by comparing two programmable/selectable results (programmable sample period; S8, S9, programmed recent value Fy, S11, and programmed historic value, S12) from the stored products (S7) from Eq (2-2), i.e., by calculating (change calculation ΔF, step S10)

ΔF=Fy−Fx Eq (2-3):

The time between two programmable/selectable results is, according to embodiments, a period of time that has elapsed between the Fx and Fy measures. It could also be a time interval in that the device is programmed to assess the difference between x and y. For example, a programmed interval of three months means that the device is programmed to compare x to y at three month intervals. Alternatively, the device may be programmed such that it continuously compares each progressive measure to a value that occurred three months earlier.

Here, particularly, negative values for ΔF may be used as an index of poor prognosis and are diagnostic of a progression of CDV/HF. The time between each compared measure in Eq (2-3) may be programmable/selectable. Multiple comparisons may also be made and displayed for additional diagnostic and statistical data. The values may be stored (stored history, S13).

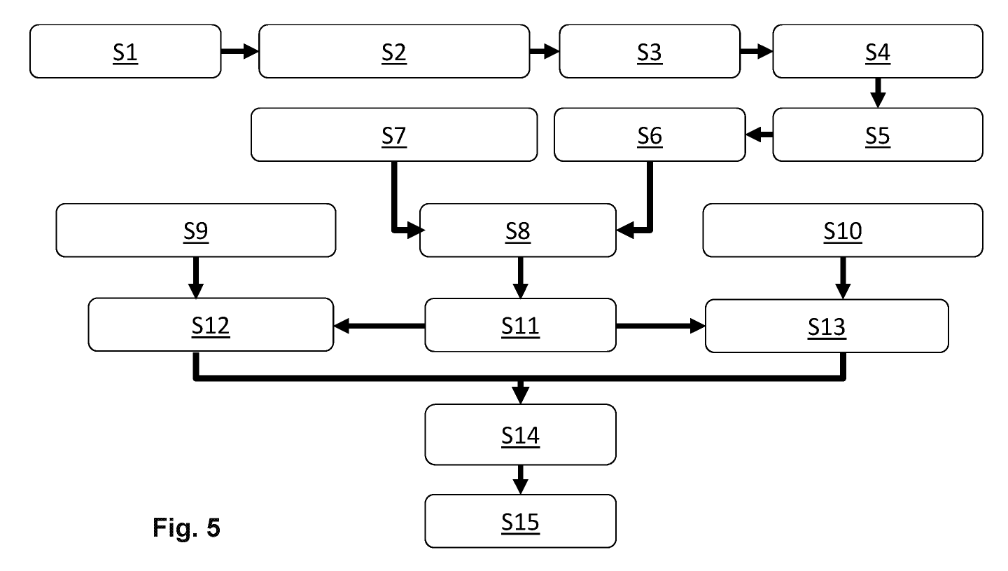

Furthermore, according to an embodiment shown in FIG. 5, tachycardia may also be tracked utilizing the variable Rd as well. However, instead of calculating a variance to the population of samples a mean R̅d̅ₒ will be generated, as shown in Equation (3-1).

Also here, as indicated in FIG. 5, the electrocardiogram signal S is detected by the detection element 2 (S1), and the processing unit 3 detects the QRS complexes (QRS detection algorithm, S2) in the received ECG signals S and uses time stamps for marking two successive R waves (QRS marker time stamp, S3), which are recorded (buffered outputs (2-sample), S4) and used to determine the corresponding R-R interval (delta of outputs, S5), which is recorded (buffered delta outputs, S6), wherein the number of recorded R-R intervals can be selected/programmed (programmable sample period, S7).

The recorded R-R intervals Rdᵢ (step S6) are averaged (R̅d̅ₒ calculation, S8) to form a mean R̅d̅ₒ of the R-R intervals according to

$${\overline{Rd}}_{o} = \frac{1}{n} ( {\sum }_{i = 1}^{n} {Rd}_{i} ) \qquad Eq ( 3 \text{-} 1 )$$

wherein the number n of samples or the sampling duration may be selectable/programmable (S7).

The determined means are recorded (R̅d̅₀ stored history, S11) to have a history of means and differences ΔRd are determined (ΔRd calculation, S14) according to

ΔRd=R̅d̅_{y}−R̅d̅ₓ Eq (3-2):

using programmed values (S12, S13), namely a more recent value R̅d̅_̅{̅y̅}̅ and an earlier value R̅d̅ₓ.

Particularly, the individual differences ΔRd are recorded (stored history, S15) and may be displayed graphically or as calculated by Eq (3-2).

A value smaller than zero for Eq (3-2) or a negative correlation between time and the values obtained from Eq (3-1) may be diagnostic of worsening CVD/HF.

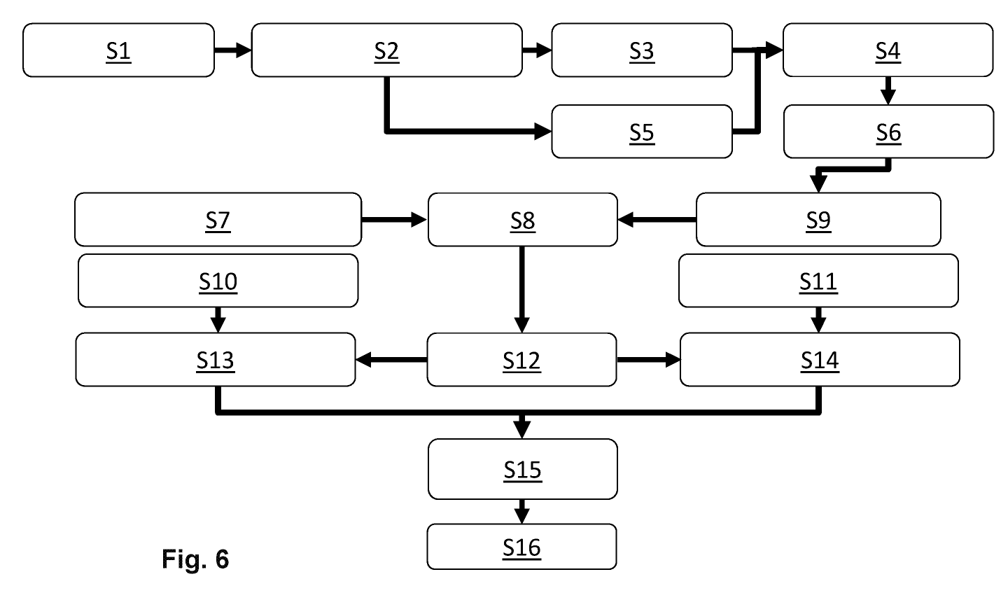

Furthermore, according to yet another embodiment shown in FIG. 6, Q-T elongation may be tracked utilizing formulae identical to Eqs. (3-1) and (3-2) by replacing the variable Rd with the measured duration between the Q to the T waves (Q-T) (cf. FIG. 1).

For this, according to FIG. 6, the electrocardiogram signal S is detected by the detection element 2 (S1), and the processing unit 3 detects the Q and T complexes (QT detection algorithm, S2) in the received ECG signals S and uses time stamps for marking a Q wave and successive T wave (Q marker time stamp, S3, and T marker time stamp, S5), which are recorded (buffered outputs, S4) and used to determine the corresponding Q-T interval QTᵢ (QT duration, S6), which is recorded (buffered QT duration outputs, S9). From these Q-T durations, a mean is determined (Q̅T̅ₒ calculation, S8) according to

$${\overline{QT}}_{o} = \frac{1}{n} ( {\sum }_{i = 1}^{n} {QT}_{i} ) \qquad Eq ( 4 \text{-} 1 )$$

wherein the number n of recorded Q-T intervals can be selected/programmed (programmable sample period, S7).

Particularly, determination of the individual Q-T interval may be most easily implemented by measuring from the peak (absolute maximum for each complex) or preferably through other methods such as a start or end detection method. In the instance the Q wave is not present or has poor resolution, the P-wave or R-wave may be utilized as a non-superior alternative.

Furthermore, the determined means are recorded (Q̅T̅ₒ stored history, S12) so as to have a history of the means and differences ΔQT are determined (ΔQT calculation, S15) according to

ΔQT=Q̅T̅_{y}−Q̅T̅ₓ Eq (4-2):

using programmed values (S13, S14), namely a more recent value Q̅T̅_{y} and an earlier value Q̅T̅ₓ.

Particularly, the individual differences ΔQT are recorded (stored history, S16) and may be displayed graphically or as calculated by Eq (4-2).

A value greater than zero for ΔQT as shown in Eq (4-2) or a positive correlation between time and the equivalent values Q̅T̅ₒ obtained from Eq (4-1) may be diagnostic of a worsening of a HF/CDV (e.g. indicator of pump failure, electrical conduction changes, ischaemia, risk for arrhythmia, rhythm disturbances, or ventricular aneurysm).

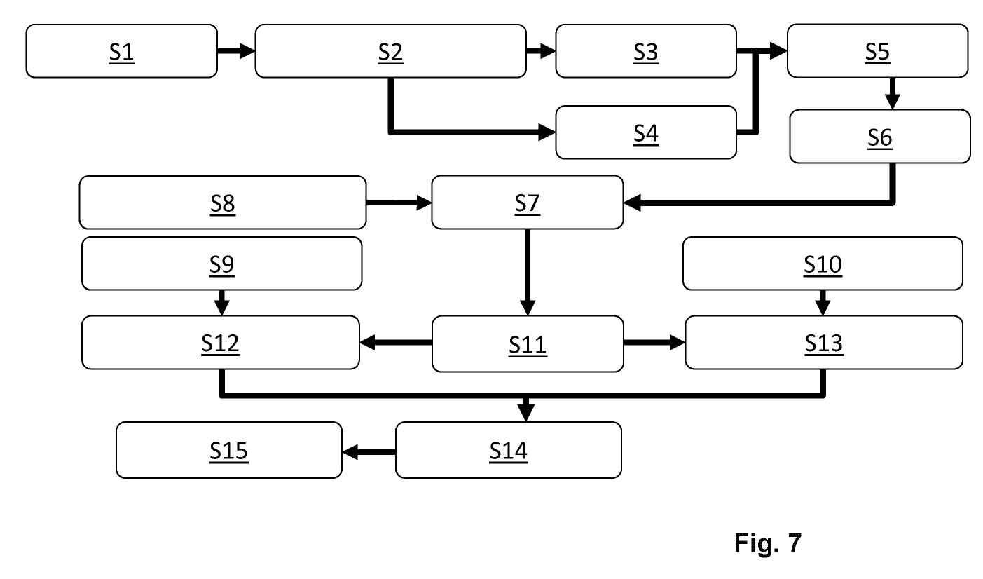

Finally, according to the embodiment shown in FIG. 7, the ratio between the absolute value of the R and S complexes, as calculated in Equation (5-1), may provide information diagnostic to RVH and LVH. In particular, devices implanted with the ECG axis oriented parallel to the midline or in a positive deviation toward the left of the midline but less than perpendicular to midline, will exhibit an increase in the R:S ratio over time in the event RVH is worsening, while the value will decrease from the time of implant if LVH is worsening. The same formulae and methodology associated with comparing values, as performed for Q-T elongation and R-R duration, may be utilized to provide this diagnostic data.

Particularly, according to FIG. 7, the electrocardiogram signal S is detected by the detection element 2 (S1), and the processing unit 3 detects the R and S complexes (RS detection algorithm, S2) in the received ECG signals S and determines their amplitudes (R amplitude, S3, and S amplitude, S4). From these amplitudes the respective R:S ratio ((R:S) calculation, S5) is determined according to

$${( R \text{:} S )}_{0} = \frac{\vert {R}_{peak} \vert}{\vert {S}_{peak} \vert} \qquad Eq ( 5 \text{-} 1 )$$

and recorded (buffered (R:S) outputs, S6). From these R:S ratios, a mean is determined ((̅R̅:̅S̅)̅ₒ calculation, S7) according to

$${\overline{( R \text{:} S )}}_{0} = \frac{1}{2} ( {\sum }_{i = 1}^{n} {( R \text{:} S )}_{i} ) \qquad Eq ( 5 \text{-} 2 )$$

wherein the number n of recorded ratios can be selected/programmed (programmable sample period, S8).

Furthermore, the determined means (̅R̅:̅S̅)̅ₒ are recorded (stored history, S11) so as to have a history of the means and differences Δ(R:S) are determined (Δ(R:S) calculation, S14) according to

Δ(R:S)=(̅R̅:̅S̅)̅_{y}−(̅R̅:̅S̅)̅ₓ Eq (5-3):

using programmed values (S12, S13), namely a more recent value (̅R̅:̅S̅)̅_{y} and an earlier value (̅R̅:̅S̅)̅ₓ.

Particularly, the individual differences Δ(R:S) are recorded (stored history, S15) and may be displayed graphically or as calculated by Eq (5-3).

Further, particularly, a value greater than zero for the difference Δ(R:S) or a positive correlation between time and the means (̅R̅:̅S̅)̅₀ points to a worsening of RVH, and a value smaller than zero for the difference Δ(R:S) or a negative correlation between time and the means (̅R̅:̅S̅)̅₀ points to a worsening of LVH.

In FIGS. 5 and 7, S9 and S10 refer to a programmable sample period, respectively. In FIG. 6, S10 and S11 refer to a programmable sample period, respectively.

1. An implantable device for monitoring a patient's electrocardiogram, the device comprising:
a detection element for acquiring electrocardiogram signals of a heart of the patient, the signals being indicative of the electrocardiogram of the patient;
a processor connected to said detection element and configured to determine from the signals at least one parameter of the electrocardiogram, the at least one parameter being an R:S ratio defined as a ratio between an absolute value of an R complex and an absolute value of an S complex.

2. The device according to claim 1, wherein said processor is configured to record a plurality of QRS amplitudes during a first sampling period and a plurality of QRS amplitudes during a later second sampling period, and to determine a net change of the QRS amplitude between the two sampling periods using a relationship
Δ̅Q̅R̅S̅=Q̅R̅S̅₍ᵢ₎−Q̅R̅S̅₍ᵢ₋ₜ₎,
wherein Q̅R̅S̅₍ᵢ₎ is an average QRS amplitude of the later second sampling period and Q̅R̅S̅₍ᵢ₋ₜ₎ is an average QRS amplitude of the first sampling period.

3. The device according to claim 2, wherein said processor is configured to determine and record a plurality of net changes of the QRS amplitude as a function of time.

4. The device according to claim 3, wherein said processor is configured to determine and record the plurality of net changes of the QRS amplitude from a time at which the implantable device is first implanted and started until a time at which the net change of the QRS amplitude has been determined most recently.

5. The device according to claim 1, wherein said processor is configured to determine and record a plurality of R-R durations.

6. The device according to claim 5, wherein said processor is configured to determine a measure of the plurality of R-R durations and said processor is further configured to determine a variance S_{(Rd)}² of the measure using a relationship
$${S}_{( Rd )}^{2} = \frac{1}{n - 1} {\sum }_{i = 1}^{n} {( {Rd}_{i} - \overline{Rd} )}^{2} ,$$
wherein n is a number of recorded R-R durations, Rdᵢ is an i^{th} recorded R-R duration, and R̅d̅ is the measure.

7. The device according to claim 6, wherein the measure is a mean of the plurality of R-R durations.

8. The device according to claim 6, wherein said processor is configured to determine and record a plurality of variances of measures of R-R durations as a function of time.

9. The device according to claim 8, wherein said processor is configured to determine and record at least one or a plurality of F values according to
$$F = \frac{{S}_{Rd ( α )}^{2}}{{S}_{Rd ( β )}^{2}} ,$$
wherein S_{Rd(α)}² is a variance of the plurality of variances that has been determined more recently than a variance S_{Rd(β)}², which is also a variance of the plurality of variances.

10. The device according to claim 9, wherein said processor is configured to determine and record a difference ΔF between two of the recorded F values Fx and Fy according to ΔF=Fy−Fx, wherein Fx has been determined and recorded before Fy.

11. The device according to claim 10, wherein said processor is configured to determine and record a plurality of differences of recorded F values.

12. The device according to clam 1, wherein said processor is configured to determine and record a plurality of Q-T durations.

13. The device according to claim 12, wherein said processor is configured to determine a mean of the plurality of Q-T durations according to
$${\overline{QT}}_{o} = \frac{1}{n} ( {\sum }_{i = 1}^{n} {QT}_{i} ) ,$$
wherein n is a number of recorded Q-T durations, and QTᵢ is an i^{th} recorded Q-T duration.

14. The device according to claim 12, wherein said processor is configured to determine and record a plurality of means of Q-T durations as a function of time.

15. The device according to claim 14, wherein said processor is configured to determine and record a difference ΔQT between two of the recorded means Q̅T̅ₓ and Q̅T̅_{y} according to ΔQT=Q̅T̅_{y}−Q̅T̅ₓ, wherein Q̅T̅ₓ has been determined and recorded before Q̅T̅_{y}.

16. The device according to claim 15, wherein said processor is configured to determine and record a plurality of differences ΔQT between recorded means.

17. The device according to claim 1, wherein said processor is configured to determine and record a plurality of R:S ratios.

18. The device according to claim 17, wherein said processor is configured to determine a mean of said plurality of R:S ratios according to
$${\overline{( R \text{:} S )}}_{0} = \frac{1}{n} ( {\sum }_{i = 1}^{n} {( R \text{:} S )}_{i} ) ,$$
wherein n is a number of recorded R:S ratios, and (R:S)ᵢ is an i^{th} recorded R:S ratio.
